(19) United States
(12) Patent Application Publication
(10) Pub. No.: US 2019/0105161 A1
(43) Pub. Date: Apr. 11, 2019

(54) FLEXIBLE CONSTRAINED LINER FOR HIP PROSTHESIS

(71) Applicant: Flexable Orthopedics Ltd., Jerusalem (IL)

(72) Inventors: Ron Holiday, Jerusalem (IL); Moshe Ben-David, Tel Aviv (IL); Ehud Lehavi, Motsa Elit (IL)

(73) Assignee: Flexable Orthopedics Ltd., Jerusalem (IL)

(21) Appl. No.: 16/209,996

(22) Filed: Dec. 5, 2018

Related U.S. Application Data
(63) Continuation-in-part of application No. PCT/US2017/036078, filed on Jun. 6, 2017.
(60) Provisional application No. 62/346,858, filed on Jun. 7, 2016.

(51) Int. Cl. A61F 2/34

(52) U.S. Cl. CPC A61F 2/34; A61F 2002/30069

(57) ABSTRACT

Provided are improved hip prosthetic devices that reduce the risk of hip dislocation upon performing artificial hip replacement. The improved hip prosthetic devices comprise a flexible bumper configured to extend from the acetabular component and prevent contact between the femoral neck and the acetabular component. The bumper may be an acetabular capsule inserted in collaboration with a standard hip prosthetic device, and attached to a standard acetabular liner. The capsule has a bumper region that cushions contact between the femoral neck and the acetabular component, thereby preventing impingement and dislocation, both common in patients in which known hip prosthetic devices are used. Alternatively, an improved acetabular liner comprises an integral bumper which prevents impingement of the femoral neck and acetabular components, thereby reducing dislocation risk and securing the femoral head within the acetabular liner.

### CROSS REFERENCE TO RELATED APPLICATIONS

This application is a continuation-in-part application which claims priority from PCT application PCT/US2017/036078, filed on Jun. 7, 2017, which claims priority under 35 U.S.C. § 119(e) from U.S. Provisional Application Ser. No. 62/346,858 filed on Jun. 7, 2016 both of which are hereby incorporated by reference in their entirety.

### TECHNICAL FIELD OF THE INVENTION

Embodiments of the invention relate to flexible constrained liners and adjunct devices to be used with liners for hip prosthesis.

### BACKGROUND OF THE INVENTION

Hip replacement surgery (also known as hip arthroplasty) is a procedure in which damaged parts of a human hip joint are removed from a patient and subsequently replaced with an artificial hip replacement. An artificial hip replacement typically comprises metal and polymeric components, also known as a hip prosthesis. A healthy hip joint is a “ball and socket” type of joint in which a part of the femur (thighbone) known as the femoral head acts as a ball which rotates within the acetabulum, which is a socket-like structure of a hip. A hip prosthesis typically comprises an acetabular prosthesis cemented or otherwise fused to the hip bone, acting as a socket. The acetabular prosthesis typically comprises a metal acetabular component which is cemented or otherwise joined to the pelvic bone, and a polymeric liner which is permanently affixed to the metal acetabular component. An additional component of the prosthesis is the femoral prosthesis, which typically comprises a ball-shaped femoral head, a femoral stem, which is typically fused into the femur, and a femoral neck, which connects the ball-shaped femoral head to the femoral stem. The femoral head is introduced into the acetabular liner and rotates within the cavity defined by the acetabular liner.

Dislocation is a medical condition in which the femoral head prosthesis is forced out of its housing within the liner of the acetabular component, and becomes removed and distanced from the cavity defined by the acetabular liner. Dislocation is one of the common complications of hip replacement surgery and is usually corrected in a process known as “revision”. Even in instances when revision is not required, dislocation can be associated with sudden acute pain, functional impairment, and soft tissue damage. Moreover, dislocation can be devastating to a patient's confidence in his or her hip replacement and in his or her surgeon.

A hip is considered to be dislocated when an intervention is required to relocate the hip. A partial dislocation known as subluxation of the hip covers a broad spectrum of clinical situations, ranging from a hip that produces only the occasional painless noise to the femoral prosthesis that frequently comes in and out of the acetabular component with a feeling of pain and instability.

The risk of dislocation has a major effect on patients' quality of life, as it often prohibits them from squatting, crossing their legs, sleeping on their sides, bending over, or even sitting on a low seat. While these precautions lessen the risk of dislocation, they do not absolutely prevent it, as dislocation remains a common issue, even for patients who attempt to avoid motions that potentially lead to dislocation.

### SUMMARY OF THE INVENTION

Embodiments of the invention provide improved hip prosthetic devices that reduce the risk of hip dislocation upon performing artificial hip replacement. The improved hip prosthetic devices comprise a flexible bumper configured to extend from the acetabular component and prevent contact between the femoral neck and the acetabular component. In an embodiment of the invention, the bumper is in an acetabular capsule and can be inserted in collaboration with a standard hip prosthetic device, and attached to a standard acetabular liner. The capsule has a bumper region that cushions contact between the femoral neck and the acetabular component, thereby preventing impingement and dislocation, both common in patients in which prior art hip prosthetic devices are used. In an embodiment of the invention, an improved acetabular liner comprises an integral bumper which prevents impingement of the femoral neck and acetabular components, thereby reducing dislocation risk and securing the femoral head within the acetabular liner.

Prosthetic devices according to the embodiments of the invention further comprise a roof having an aperture sized to be large enough for a femoral neck to freely move along a medial region of its range of motion within the aperture without contacting the roof. At lateral extremes of the range of motion of the femoral neck, the femoral neck contacts the roof. The aperture may be sized to be smaller than the diameter of the femoral head and thereby act as a restraint to hold the femoral head in the vicinity of the aperture defined by the acetabular liner, thereby preventing dislocation.

Provided are hip prosthetic stabilization capsules for use in conjunction with a femoral head, neck and stem prosthesis and with an acetabular prosthetic component, the capsule comprising:
a roof having an aperture, the aperture at its narrowest axis having a diameter smaller than the diameter of the femoral head and greater than that of the femoral neck; and
a cylindrical, compressible bumper region comprising a plurality of windows, the bumper region attached at its one end to the acetabular component and at its other end to the roof.

### BRIEF DESCRIPTION OF THE DRAWINGS

Non-limiting examples of embodiments of the invention are described below, with reference to a figure attached hereto. Dimensions of components and features shown in the figures are chosen for convenience and clarity of presentation and are not necessarily shown to scale.

FIGS. 1A and 1B depict prior art hip prosthesis and associated increased risk of dislocation as a result of impingement;

FIGS. 2A and 2B depict an acetabular liner according to an embodiment of the invention in a side and top view, respectively;

FIGS. 2C, 2D and 2E depict views of a hip prosthesis joint according to an embodiment of the invention;

FIGS. 3A and 3B depict a capsule according to an embodiment of the invention in a perspective view and a side view, respectively;

FIG. 4A depicts an exploded view of acetabular liner system according to an embodiment of the invention, comprising an acetabular liner, a capsule, and a ring for joining the capsule with the acetabular liner;

FIG. 4B depicts a perspective view of a hip prosthesis joint according to an embodiment of the invention; and

FIGS. 5A, 5B and 5C depict cross-sectional views of hip prosthesis joints according to an embodiment of the invention; and

FIGS. 6A and 6B depict a capsule according to an embodiment of the invention in a top view and a side view, respectively.

### DETAILED DESCRIPTION OF THE INVENTION

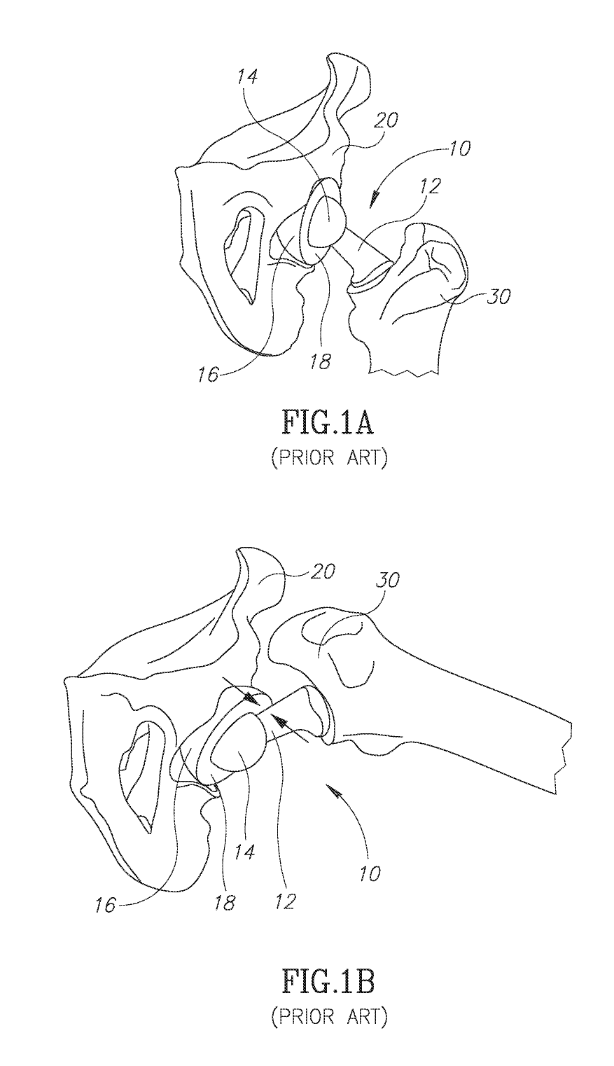

As mentioned above, prior art prosthetic devices are associated with high risk of dislocation. A prior art hip prosthetic device 10 is depicted in FIG. 1A and FIG. 1B. FIG. 1A shows a hip prosthetic device installed in a patient in a normal configuration. Hip prosthetic device 10 comprises a femoral neck 12, a femoral head 14 and a femoral stem (not shown) imbedded into a femur 30. Hip prosthetic device 10 further comprises a liner 18, lining an acetabular component 16. Acetabular component 16 is affixed, via cement or pressure fit to a patient's pelvic bone 20. Femoral head 14 rotates within liner 18 as a patient moves his or her femur 30 relative to his or her pelvic bone 20 in everyday activities such as walking, sitting and standing.

Reference is now made to FIG. 1B which shows hip prosthetic device 10 in an impinging configuration, wherein femoral neck 12 is moved to an angle of nearly 180 degrees relative to the plane formed by the circumference of acetabular liner 18. The patient in which hip prosthetic device 10 has been inserted is positioned in a position in which his or her thigh is positioned relative to his or her hip at an extreme of his or her range of motion. Femoral neck 12 contacts acetabular component 16 and/or liner 18 as indicated by arrows. When force is applied in the direction of the arrows, stress increases on femoral head 14, and dislocation of femoral head 14 from liner 18 becomes more likely.

Impingement between a metal femoral neck 12 and a rim of liner 18 can damage the polymeric liner 18 both at the site where femoral neck 12 contacts the rim, and the regression where femoral head 14 escapes from polymer liner 18 bore.

When external load challenges on a hip prosthetic are high, the resistive moment within the polymer can exceed the yield strength of the polymer and, with chronic impingement, can lead to polymer damage through increased wear and/or cracking of the liner with subsequent implant failure. Liner cracks are associated with some degree of impingement damage and oxidation. Impingement is prevalent even in hip replacements with no history of dislocation.

Impingement between the prosthetic components and soft tissue on bone can lead to instability of the hip prosthetic device. In addition to prosthetic instability, pain is a common consequence of impingement.

The mechanical explanation of dislocation is defined as follows: Following the impingement of femoral neck 12 and liner 18, subluxation will start to occur causing prosthetic femoral head 14 to lift out of its socket, eventually leading to the center of the prosthetic femoral head 14 to lift above the perimeter of the prosthetic acetabular socket causing frank dislocation. The impingement point may act as a fulcrum around which femoral head 14 is levered out of the socket.

Previous attempts have been made to overcome hip prosthetic weaknesses associated with impingement. For example, modifying a liner to be “deeper” to engage more surface area of a femoral head has been attempted. However, in such devices, the deep socket further limits the range of joint motion, limiting daily activities. Although such a prosthetic may limit the risk of dislocation, in the cases that dislocation occurs it is necessary to return the ball to the socket surgically.

US Patent application 2007/0276364, U.S. Pat. Nos. 6,923,833, 5,514,182 and U.S. Pat. No. 6,228,122 describe prosthetic devices designed to reduce hip prosthetic dislocation, having fastening means for fastening to the femoral neck. A disadvantage of the prosthetic devices provided in this application is that the devices which are intended to prevent dislocation are connected to the femoral neck, requiring a patient to exert more force than needed during normal activities in order to overcome the force exerted by the mesh.

It has been found that in many prior art hip replacement prostheses, there is a tradeoff between stability and range of motion. Changes of structure performed on a hip replacement prosthesis which increase stability, or maximum resisting moment, have the effect of lessening the allowable range of motion to impingement and also can lead to dislocation. In addition, changes which allow more range of motion generally lead to less stability, or lower resisting moment being need to dislocate.

Embodiments of the present invention relate to hip prostheses that provide increased stability and increased range of motion.

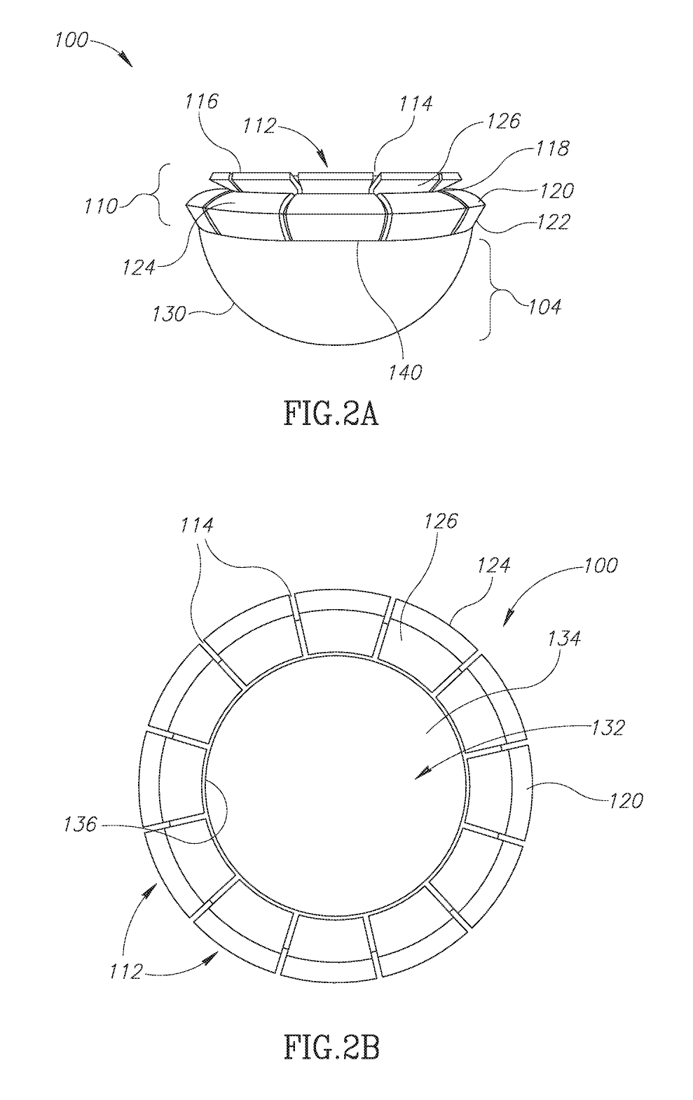

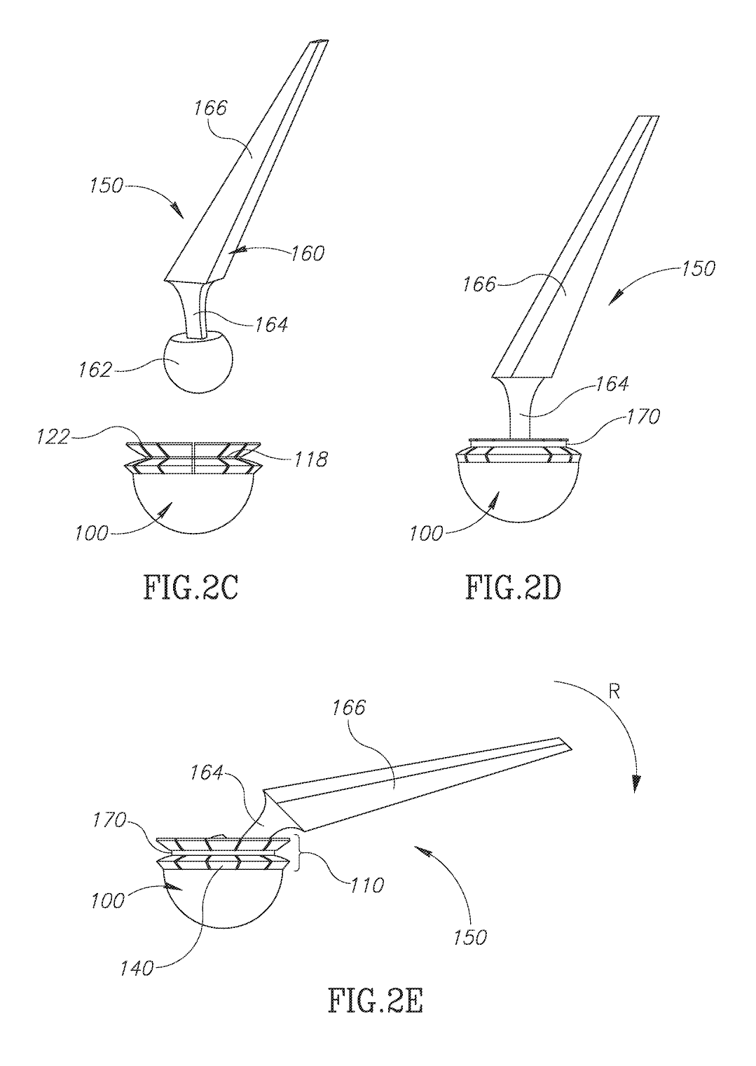

Reference is now made to FIGS. 2A and 2B which respectively depict side and top views of an acetabular liner 100 according to an embodiment of the invention. Acetabular liner 100 is configured to be used to line a metal acetabular component (not shown) which is cemented or otherwise joined to the pelvic bone. Acetabular liner 100 comprises a hemispherical region 104 and a bumper region 110.

Hemispherical region 104 comprises an outer surface 130 and an inner surface 134, defining a liner receiving area 132. Liner receiving area 132 is configured to receive a femoral head prosthesis. Circumference of liner receiving area is substantially equal to circumference of the femoral head for which it is configured to receive. Outer surface 130 is configured to be affixed to a metal acetabular component.

Bumper region 110 extends from margin 140 at the circumference of hemispherical region 104. Bumper region 110 comprises tabs 112 and apertures 114, separating between tabs 112. Tabs 112 are accordion-like in structure and comprise a lower tab element 122, a middle tab element 124 and a terminal tab element 126. Tabs 112 comprise joints at margin 140, outer lip 120 and groove 118. Lower tab element 122 extends circumferentially outwards from hemispherical region 104 at margin 140. Middle tab element 124 extends circumferentially inwards from lower tab element 122 at outer lip 120. Terminal tab element 126 extends circumferentially outwards from middle tab element 124 at groove 118. Terminal element 126 comprises terminal edge 116. Inner circumference 136 is defined by the inner surface of tabs at groove 118, and is a roof aperture defined by terminal element 126

Terminal element 126 is a roof of acetabular liner 100.

Acetabular liner 100 comprises twelve tabs 112 and twelve apertures 114. Alternative embodiments of acetabular liners according to the invention may comprise n tabs and n apertures wherein n is 1 or more.

In acetabular liner 100, tabs 112 each comprise 3 tab elements (122, 124, and 126.) Tabs according to alternative embodiments of the invention may each comprise between 1 and 1000 tab elements.

Acetabular liner 100 may be formed from a single polymer or a combination of polymers. It may be formed using a mold in an injection molding process or by 3-dimensional printing. The polymer used is preferably hard yet elastic polymer. The hardness of the polymer is preferably between 70-100 in the Shore A scale, or between 30-60 in the shore D scale. The polymer used may be selected from one or more than one of the polymers listed in Table 1, all available from DSM Biomedical, Exton, Pa. USA.

TABLE 1

| Polymer trade name | Polymer description |
|---|---|
| Bionate ® 80A, 90A | Thermoplastic Polycarbonate |
| or 55D | Polyurethane (PCU) |
| BioSpan ® SPU | Segmented Polyurethane |
| Carbosil ® TSPCU | Thermoplastic Silicone- |
| | Polycarbonate-urethane |
| Elasthane ™ TPU | Thermoplastic polyether- |
| | urethane |
| Pursil ® TSPU | Thermoplastic Silicone- |
| | Polyether-Urethane |

Reference is now made to FIGS. 2C, 2D and 2E depicting a detached side view (FIG. 2C), and a connected side view in normal position (FIG. 2D), and a connected side view in extended position (FIG. 2E) of a prosthetic hip joint 150 according to an embodiment of the invention. Hip joint 150 comprises a femoral prosthesis 160 and an acetabular liner 100, configured to be affixed to a metal acetabular component fused to the pelvic bone (both metal acetabular component and pelvic bone not shown).

Femoral prosthesis 160 comprises a femoral head 162, a femoral neck 164 and a femoral stem 166. In FIG. 2C, femoral head 162 has not yet been introduced into acetabular liner. During installment of femoral prosthesis 160 in a hip replacement surgery, acetabular liner 100 is affixed to an acetabular component (not shown) that has been cemented to a human pelvic bone. Femoral head 162 and femoral neck 164 are together pushed in the direction of acetabular liner 100 so that tabs 112 move circumferentially outward. Upon introduction into liner receiving area 132 (see FIG. 2B), a securing tie 170 is introduced into groove 118, securing tabs 112 around femoral head 162 as shown in FIG. 2D.

Securing tie 170 may comprise ultra-high molecular weight polyethylene. An exemplary type of securing tie 170 may be made from Dyneema® made by DSM Biomedical, Exton, Pa. USA. Securing tie 170 may be wrapped around groove 118 once or more than once. Securing tie 170 may be tied with a conventional knot or with a one-directional locking system.

Upon tightening securing tie 170, femoral head is secured in liner receiving area 132 and femoral prosthesis 160 is movable over a substantial range of motion without femoral neck 164 contacting acetabular liner 100. Preferably, the range of motion over which femoral prosthesis 160 is movable without contact of femoral neck 164 with acetabular liner 100 is about 130 degrees. Upon tightening of securing tie 170, inner circumference 136 (see FIG. 2B) of groove 118 is smaller than the circumference of the spherical element of femoral head 162. Motion of femoral prosthesis 160 in the lateral direction indicated by arc R in FIG. 2E contacts femoral neck 164 with roof of acetabular liner 100 at bumper region 110, thereby compressing bumper region 110 and providing resistance to further motion of femoral neck 164.

Upon tightening securing tie 170, acetabular liner may resist forces of dislocation up to 100 Nm.

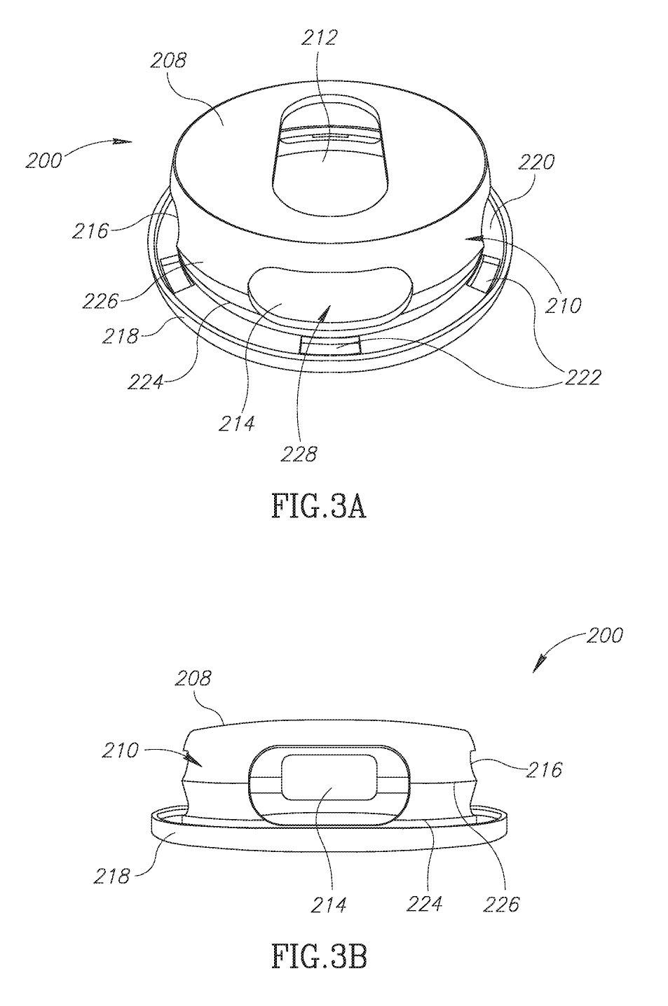

Reference is now made to FIGS. 3A and 3B depicting a capsule 200 according to an embodiment of the invention in a perspective view (3A) and a side view (3B).

Capsule 200 comprises a roof 208, a plurality of legs 210, a roof aperture 212, a minor window 214, a major window 216, an external lip 218, a ring groove 220, a plurality of fastening apertures 222, a lower flexing fold 224, and an upper flexing fold 226.

Capsule 200 may be formed using the same methods and from the same types of polymers described in connection with construction of acetabular liner 100. Alternatively, capsule 200 may be formed using a biodegradable polymer, or a combination of a plurality of biodegradable polymers. A biodegradable polymer is a polymer which is stable for long term at storage conditions, but upon introduction into a human body, degrades over the course of months. The conditions in the human body such as presence of water or water-based liquids, and temperature of about 37□ may induce degradation of biodegradable polymers.

As mentioned previously, a risk of hip replacement surgery is dislocation. The likelihood of dislocation is higher during the time period after a hip replacement surgery. As time passes from the hip replacement surgery, the risk of dislocation decreases. The highest risk of dislocation is during the first three months following surgery. Risk is moderate between 3-12 months. After 12 months post-surgery, the risk of dislocation decreases. For this reason a biodegradable capsule may be advantageous to reduce risk of dislocation for the first 3-12 months after surgery, but may degrade after that time, when risk for dislocation is reduced.

Capsule 200 may be formed from a biodegradable polymer and may be configured to begin to biodegrade from 3 months after introduction into a human body. Alternatively, the biodegradable polymer and may be configured to begin to biodegrade from 6 months after introduction into a human body. Alternatively, the biodegradable polymer and may be configured to begin to biodegrade from 12 months after introduction into a human body.

According to an embodiment biodegradable polymers which may be used to form capsules according to some embodiments are selected from the group consisting of: collagen; lactide/glycolide copolymer; polyester comprising glycolide, caprolactone, trimethylene carbonate and lactide; glycolide and epsilon-caprolactone copolymer; glycolide homopolymer; glycolide, polyester comprising dioxanone and trimethylene carbonate; glycolide and trimethylene carbonate copolymer; poly glycolic acid; and polyester of p-dioxanone; poly (4-hydroxybutyric acid).

Capsule 200 is sized so that the circumference of ring groove 220 is similar in size to the acetabular liner to which capsule 200 is mated.

Capsule 200 further comprises an inner volume 228 defined by legs 210 and roof 208. Inner volume 228 is large enough to allow a femoral head to rotate freely within it, while femoral head is contained within an acetabular liner mated to capsule 200 via ring groove 220.

Capsule 200 comprises two minor windows 214 and two major windows 216, which are apertures. Capsule 200 also comprises four legs 210 connecting roof 208 with ring groove 220. Roof 208 with ring groove 220 lie in substantially parallel planes. Minor windows 214 are smaller in size than major windows 216. Minor windows 214, major windows 216 and legs 210 together define a cylindrical curved surface acting as a bumper area. The area of minor and major windows 214 and 216 together are larger than the legs 210 area. Alternate embodiments of the invention may comprise between 1 and 1000 legs, preferably between 3 and 12 legs. Minor windows 214 and major windows 216 are each stadium shaped. Alternate embodiments of the invention may comprise windows having other shapes such as square, rectangular, oval and elliptical.

Roof aperture 212 is stadium shaped. Alternate embodiments of the invention may comprise a roof aperture having a rectangular, oval or elliptical shape.

Legs 210 are each constructed having two folds for added flexibility, lower flexing fold 224, and upper flexing fold 226. As seen in FIG. 3B, legs 210 are accordion-shaped. Alternate embodiments of the invention comprise legs having between 1 and 100 folds.

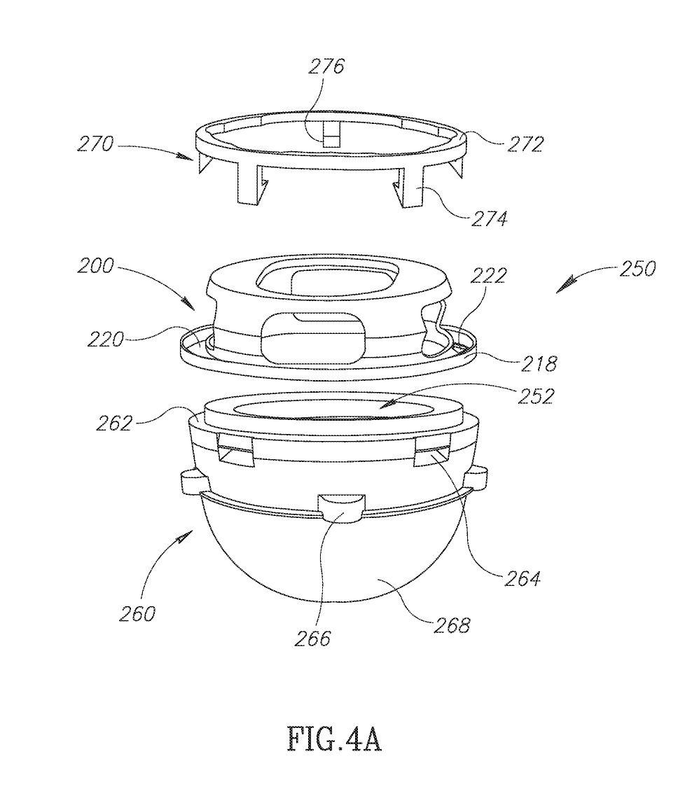

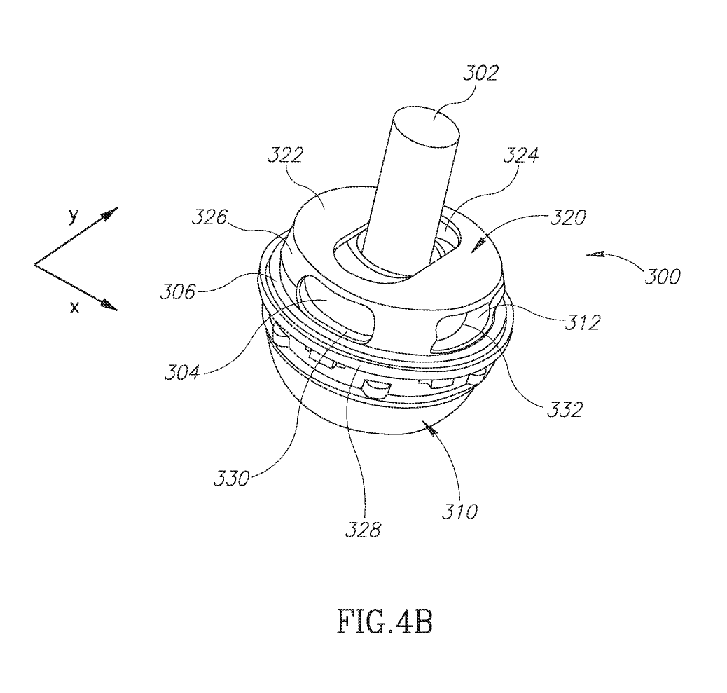

Reference is now made to FIG. 4A which depicts an exploded view of an acetabular liner system 250 comprising an acetabular liner 260, a capsule 200, and a mating ring 270.

Acetabular liner 260 comprises a rim notch 262, a plurality of indentations 264, a plurality of guide protrusions 266 and outer bowl surface 268. Acetabular liner 260 further comprises a receiving area 252.

Mating ring 270 comprises a hoop 272 and a plurality of fingers 274 and a plurality of mating protrusions 276, each extending circumferentially inwards from fingers 274.

During the operation of acetabular liner system 250 in a hip replacement surgery, acetabular liner 260 is inserted into an acetabular component (not shown) which has been affixed to a patient's hip, by inserting outer bowl surface 268 into the acetabular component, until guide protrusions 266 contact the circumference of the rim of the acetabular component. Acetabular liner 260 is affixed to acetabular component by applying pressure, optionally via tapping with a hammer. Femoral head, which has been attached to femoral neck (both not shown) is introduced into receiving area 252 of acetabular liner 260.

Capsule 200 is placed on acetabular liner 260 by aligning capsule 200 with rim notch 262. Capsule 200 is aligned with acetabular liner 260 so that fastening apertures 222 correspond to indentations 264. Mating ring 270 is then used to secure capsule 200 to acetabular liner 260 by introducing fingers 274 into fastening apertures 222. Hoop 272 is introduced into ring groove 220 between external lip 218 and legs 210. Fingers 274 extend over perimeter of acetabular liner 260 and mating protrusions 276 snap fit into indentations 264.

Some embodiments relate to alternate methods for securing capsule 200 to acetabular liner 260. An adhesive may be applied to capsule 200 and/or to acetabular liner 260 at the areas in which they contact each other. During operation of the acetabular liner system in such an embodiment, a femoral head may be introduced into an acetabular component, and subsequently, adhesive may be applied to a capsule and/or to an acetabular liner. The capsule and acetabular liner are then contacted to each other, and the adhesive prevents separation of the capsule from the acetabular liner.

Adhesive which may be used according to an embodiment is selected from the group consisting of a polyurethane adhesive, cyanoacrylate adhesive, an ultraviolet-activated adhesive, an acrylic adhesive, a silicone adhesive and an epoxy adhesive. The adhesive may be a biodegradable adhesive.

Reference is now made to FIG. 4B, showing parts of an assembled prosthetic hip joint 300 according to an embodiment of the invention. Hip joint 300 comprises a prosthetic femoral neck 302, a prosthetic femoral head 304, a capsule 320, a mating ring 306 and an acetabular liner 310.

Capsule 320 comprises a roof 322, a roof aperture 324, legs 326, external lip 328, minor windows 330, and major windows 332.

Acetabular liner 310 comprises a perimeter 312, a rim stopper 314.

Prosthetic hip joint 300 may be assembled as described with reference to acetabular liner system 250. As shown in figure, roof aperture 324 is larger than the circumference of femoral neck 302, at the section of femoral neck 302 which contacts roof 322 upon lateral motion. Motion of femoral neck 302 within roof aperture 324 both along the x-axis and along the y-axis may proceed without contact between femoral neck 302 and roof aperture 324. When prosthetic hip joint 300 is installed within a patient and femoral neck 302 moves during normal motion of the patient though rotation of femoral head 304 within liner 310, no contact is made between femoral neck 302 and capsule 320. As a result, most of the patient's regular activities are not restricted by pressure between femoral neck 302 and capsule 320. Motion for about 80-100 degrees along the y axis without contact between femoral neck 302 and roof aperture 324 is possible. At extreme ranges of motion along the x or y axis, femoral neck 302 contacts roof 322, providing relatively minor resistance. As femoral neck 302 continues along the y-axis, legs 326 are compressed, closing windows 330 and 332, providing increased resistance to motion of femoral neck 302 relative to liner 310. Capsule 320 acts as a bumper absorbing pressure and preventing impact between femoral neck 302 and perimeter 312, thereby preventing impingement and associated damage.

Table 2 below describes ranges, in degrees, of angles of various types of motion capable in patients in which prosthetic hip joint 300 is installed. The patient may move at these angles of motion without risk of dislocation.

TABLE 2

| | Range of | |
|---|---|---|
| | motion for | Full |
| | stages of | range |
| Motion type | motion | of motion |
| Flexion | 100-190 | 120-240 |
| Extension | 20-50 | |
| Abduction | 45-50 | 60-75 |
| Adduction | 15-25 | |
| Internal rotation | 45-50 | 90-100 |
| External rotation | 45-50 | |

With regard to sizing of roof aperture 324, it should be noted that the flexibility of material from which capsule 320 is formed should be taken into account, as upon lifting of femoral head 304 from socket, roof 322 may be stretched, as well as aperture 324. In order to prevent dislocation, roof aperture 324 may be configured to be no wider along its x-axis than 5-25% less than the diameter of femoral head.

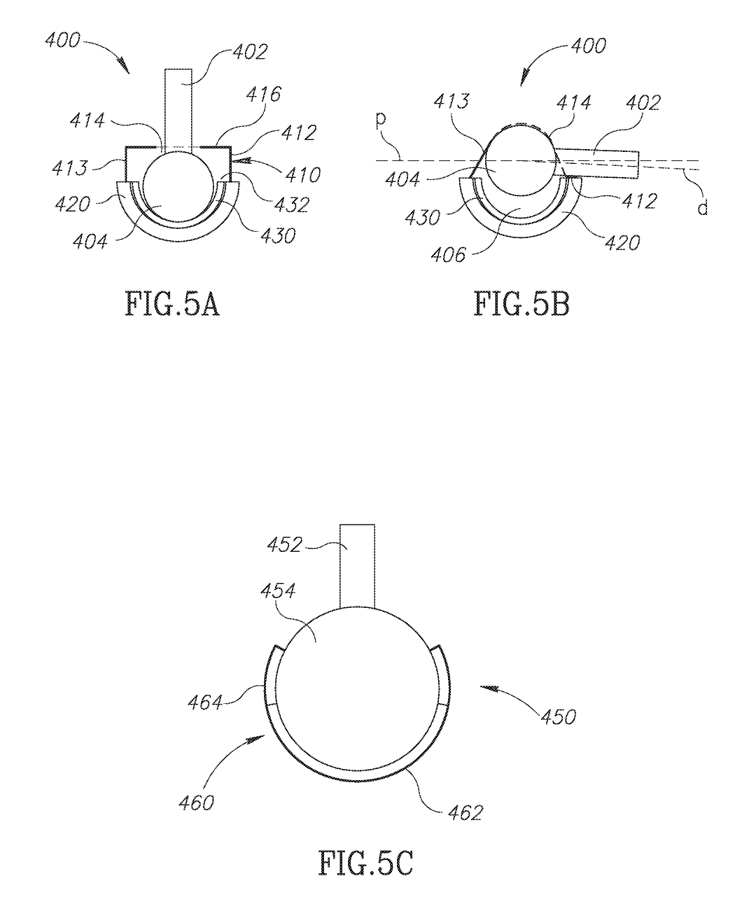

Reference is now made to FIGS. 5A and 5B which depict a cross-sectional view of a prosthetic hip joint 400 according to embodiments of the invention. Hip joint 400 comprises a femoral neck 402, a femoral head 404, a capsule 410, an acetabular component 420, and an acetabular liner 430, having a circumference 432. Capsule 410 is depicted to show its outer perimeter for purposes of clarity. During its operation, femoral neck 402 is connected to a femoral stem (not shown), which is embedded into a femur (not shown). Capsule 410 comprises bumper regions 412 and 413, and roof aperture 414. FIG. 5A depicts hip joint 400 in a position representing standard range of motion of the hip joint. Femoral head 404 is abutting acetabular liner 430 and rotatable therein. Femoral neck 402 is contained within roof aperture 414 without contacting roof 416. Femoral neck 402 is movable over a significant range of motion preferably between 80 and 130 degrees, without contacting roof 416.

FIG. 5B shows hip joint 400 at a lateral extreme position of its motion. Femoral neck 402 moves towards circumference 432 of acetabular liner 430, compressing bumper region 412 at one end of capsule 410, while stretching bumper region 413 at the opposite end of capsule 410. Compressed end of bumper region 412 prevents impingement of acetabular liner 430. As pressure is increased on femoral neck 402, femoral neck 402 continues to move towards compressed bumper region 412, femoral head 404 undergoes lifting from a receiving area 406 defined by the inner surface of acetabular liner 430. Capsule 410 acts to restrain femoral head 404 so that femoral head 404 does not dislocate. Upon release of pressure from femoral neck 402, the elastic capsule 410 reverts femoral head 404 to receiving area 406.

FIG. 5B illustrates the range of motion of hip joint 400, which is greater than 180 degrees relative to the cross section of plane p. Plane p is a plane which runs through the center of femoral head 404 and is parallel to the plane defined by the circumference 432 of acetabular liner 430. When femoral head 404 undergoes lifting and is restrained from dislocation by capsule 410, an axis d which runs from the center of femoral head through the center of femoral neck 402 lies beyond the cross section of plane p, indicating that range of motion of hip joint 400 can be greater than 180 degrees without frank dislocation of femoral head 404 from acetabular liner 406.

Although FIGS. 5A and 5B depict advantages associated with a capsule device according to embodiments of the invention, similar advantages of preventing impingement, allowing for a range of motion of femoral neck of greater than 180 degrees, and preventing dislocation, can be obtained using an improved acetabular liner according to embodiments of the invention such as acetabular liner 100.

Reference is now made to FIG. 5C which depicts a cross-sectional view of a prosthetic hip joint 450 according to embodiments of the invention. Hip joint 450 comprises an acetabular liner 460, a femoral head 454 and a femoral neck 452. Acetabular liner 460 comprises a containment region 462 and a bumper region 464. Containment region 462 may be rigid and bumper region 464 may be flexible, constructed in a similar fashion to bumper region 110 in FIGS. 2A and 2B.

As see in FIG. 5C, containment region 462 contacts less than half of the surface area of femoral head 454. During operation of hip joint 450, femoral neck 452 approaches bumper region 464, which is then compressed, optionally, through folding of the folds which comprise the bumper region. Bumper region 464 prevents impingement and secures femoral head 454 within the confines of acetabular liner 460.

Due to compression of bumper region 464, range of motion of femoral head within acetabular liner 460 may be about 165 degrees. Alternatively, range of motion is greater than 180 degrees.

An additional benefit of the bumper according to embodiments of the invention relates to promoting bone remodeling at the femoral bone-implant interface. This may strengthen the prosthetic by preventing fracture at the proximal femur, preventing loosening and eliminating micro-movements.

In previously known hip replacement systems between 32-61% of all total hip replacement patients suffer from bone loss in the proximal femur, particularly in the peri-prosthetic region. In fact, the leading cause of hip replacement failure is aseptic loosening which is primarily caused by proximal bone density loss. One possible explanation for this phenomenon is due to the metallic stem the body-load is transferred to the distal femur causing the distal femur to densify, while the proximal femur is under-loaded causing substantial bone loss.

In previously known systems, the impingement of the prosthetic components is harmful, possibly causing third body particles, deformation of the liner which can lead to hip instability, and possibly even lead to component fracture.

Hip replacement systems according to the present invention transfer the aforementioned adverse force and pressure to the proximal femur, causing pressure at the peri-prosthetic bone-prosthetic interface. This pressure in the proximal region of the femur will cause trabecular bone growth, based on Wolff's Law and on recent research relating to Rest Inserted Load.

In hip replacement systems according to embodiments of the invention, upon approaching an impingement configuration, the bumper area absorbs some of the energy of the contact, and prolongs the force impact compared to systems without a bumper area. As a result, the pressure wave formed from the impingement has a lower frequency in systems with a bumper area. This results in even pressure distribution over the length of the bone, not merely at the contact area between the femoral stem and the bone. This lowers chance of aseptic loosening failure of the hip replacement system.

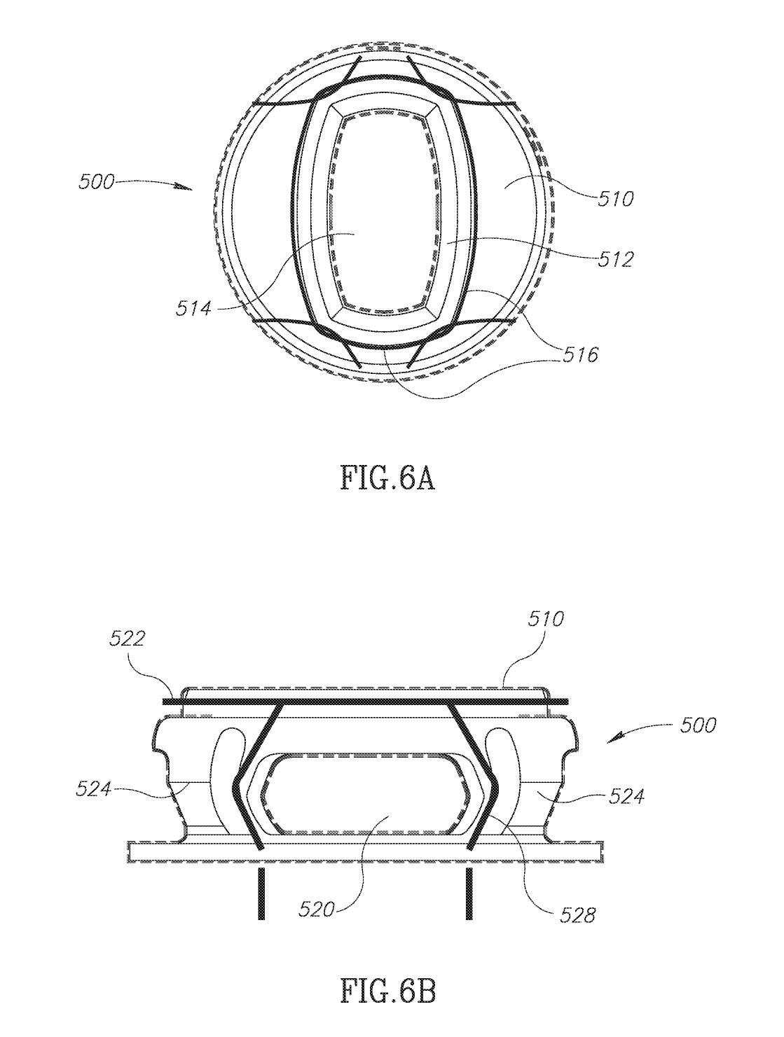

Reference is now made to FIGS. 6A and 6B which depict a capsule 500 according to an embodiment of the invention in a top view and a side view, respectively. Capsule 500 comprises a roof 510, a roof aperture 514, legs 524, windows 520, and a roof containment ring 512. Roof containment ring may be made of a polymeric material or of metal. Capsule 500 further comprises roof aperture reinforcements 516, window reinforcement 528 and roof perimeter reinforcement 522. Reinforcements 516, 528, and/or 522 may be made from a material other than the polymer which is used for making the roof and/or legs of capsule 500. Reinforcements 516, 528, and 522 may comprise ultra-high molecular weight polyethylene. An exemplary type of reinforcement may be made from Dyneema® made by DSM Biomedical, Exton, Pa. USA. Reinforcements may also comprise metal wire or other fibers. Reinforcements may be strand-like in structure. Reinforcements may be web-like or woven. Reinforcements may be strands ranging in diameter between about 0.1 and 5 mm.

Reinforcements may be introduced to acetabular liner 100 (such as depicted in FIGS. 2A and 2B). Reinforcements may be provided along perimeter of tabs 112 and/or around outer circumference of hemispherical region 104.

During operation of capsule 500, reinforcements 516, 528, and/or 522 provide additional strength and durability to capsule 500.

### EXAMPLE 1

Testing was performed on hip prosthesis systems 150 (strength) and 300 (flexibility) described above. Results of physical testing are detailed in Table 3.

TABLE 3

| Prosthetic | Lever-out torque |
|---|---|
| Acetabular liner (150) | Up to 90 Nm |
| Capsule system (300) | Up to 80 Nm |

Results in table 3 show that hip prosthesis systems according to embodiments of the invention provide increased resistance to dislocation while maintaining high flexibility and large range of motion. When compared to commercially available systems, hip prosthesis systems according to embodiments of the invention out-performed the following systems: Biomet (Freedom Liner, Ring Lock II); DePuy Synthes (Polydial, S-ROM, Durolock and Esc); Zimmer (Longevity, Epsilon Durasul, and Triology); Stryker® (Trident and Omnifit); Smith & Nephew (Reflection and R3); and Exactech® (Novation and Accumatch.)

Embodiments of the invention relate to a hip prosthetic stabilization capsule for use in conjunction with a femoral head, neck and stem prosthesis and with an acetabular prosthetic component, the capsule comprising: a roof having an aperture, the aperture at its narrowest axis having a diameter smaller than the diameter of the femoral head and greater than that of the femoral neck; and a cylindrical, compressible bumper region, attached at its one end to the acetabular component and at its other end to the roof. Optionally, the capsule is formed from an elastic polymer. Optionally, the capsule polymer is selected from the group consisting of: Thermoplastic Polycarbonate Polyurethane, Segmented Polyurethane, Thermoplastic Slicone-Polycarbonate-urethane, Thermoplastic polyether-urethane and Termoplastic Silicone-Polyether-Urethane. Optionally, the bumper region comprises accordion-like folding legs, each leg comprising between 1 and 4 folds. Optionally, the bumper region further comprises windows. Optionally, the surface area of the windows is greater than the surface area of the legs. Optionally, the capsule comprises windows having different sizes in the bumper region. Optionally, the capsule further comprises a mating region configured to mate with a circumferential area of an acetabular liner. Optionally, the mating region comprises a groove having fastening apertures, the apertures configured to contain fastening elements to attach to the circumferential area of the acetabular liner. Optionally, the capsule further comprises a circular fastening ring having fastening elements adapted to insert into fastening apertures and attach to acetabular liner. Optionally, the fastening elements snap-fit into the acetabular liner. Optionally, the roof aperture is stadium-shaped. Optionally, the capsule further comprises a reinforcement strand. Optionally, the reinforcement strand is connected to the capsule at one, or more than one of, a roof aperture, a window, or a roof perimeter. Optionally, the bumper region extends from and is an integral part of an acetabular liner. Optionally, the bumper region comprises between 4 and 20 tabs. Optionally, the tabs are accordion like tabs which extend from a femoral head receiving region to a terminal region, the tabs comprising folds. Optionally, the capsule further comprises a circumferential groove defined by tab folds. Optionally, the capsule further comprises a securing tie in a circumferential groove, configured to secure the capsule around a femoral head. Optionally, the inner surface of the tabs at the groove define an inner circumference, smaller than the circumference of the spherical element of femoral head. Optionally, the acetabular liner comprises a bumper region and a rigid containment region. Optionally, upon introduction of a femoral head into the containment region of the acetabular liner, the containment region contacts less than half of the surface area of femoral head. Optionally, when the capsule is secured around a femoral head, allows for movement of the femoral head within an acetabular liner within a medial range of motion, without contacting between the capsule roof and the femoral neck. Optionally, upon movement of the femoral head in the acetabular liner to a lateral extreme range of motion, contact is made between the capsule roof and the femoral neck. Optionally, upon lifting of the femoral head from the acetabular liner, applies pressure on the femoral head to return it to the acetabular liner.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. A hip prosthetic stabilization capsule for use in conjunction with a femoral head, neck and stem prosthesis and with an acetabular prosthetic component, the capsule comprising:
a roof having an aperture, the aperture at its narrowest axis having a diameter smaller than the diameter of the femoral head and greater than that of the femoral neck; and
a cylindrical, compressible bumper region comprising a plurality of windows, the bumper region attached at its one end to the acetabular component and at its other end to the roof.

2. The capsule according to claim 1, wherein the capsule is formed from an elastic polymer.

3. The capsule according to claim 2, wherein the capsule polymer is selected from the group consisting of: Thermoplastic Polycarbonate Polyurethane, Segmented Polyurethane, Thermoplastic Slicone-Polycarbonate-urethane, Thermoplastic polyether-urethane and Thermoplastic Silicone-Polyether-Urethane.

4. The capsule according to claim 1 wherein the capsule comprises a biodegradable material, which, upon introduction into the human body, degrades within 12 months.

5. The capsule according to claim 4, wherein the biodegradable material is selected from the group consisting of: collagen; lactide/glycolide copolymer; polyester comprising glycolide, caprolactone, trimethylene carbonate and lactide; glycolide and epsilon-caprolactone copolymer; glycolide homopolymer; glycolide, polyester comprising dioxanone and trimethylene carbonate; glycolide and trimethylene carbonate copolymer; poly glycolic acid; and polyester of p-dioxanone; poly (4-hydroxybutyric acid).

6. The capsule according to claim 1, wherein the bumper region comprises accordion-like folding legs, each leg comprising between 1 and 4 folds.

7. The capsule according to claim 1 wherein the surface area of the windows is greater than the surface area of the legs.

8. The capsule according to claim 1, comprising windows having different sizes in the bumper region.

9. The capsule according to claim 1, further comprising a mating region configured to mate with a circumferential area of an acetabular liner.

10. The capsule according to claim 1, wherein the roof aperture is stadium-shaped.

11. The capsule according to claim 1, further comprising a reinforcement strand.

12. The capsule according to claim 11 wherein the reinforcement strand is connected to the capsule at one, or more than one of, a roof aperture, a window, or a roof perimeter.

13. The capsule according to claim 1 wherein, when secured around a femoral head, allows for movement of the femoral head within an acetabular liner within a medial range of motion, without contacting between the capsule roof and the femoral neck.

14. The capsule according to claim 1, wherein, upon movement of the femoral head in the acetabular liner to a lateral extreme range of motion, contact is made between the capsule roof and the femoral neck.

15. The capsule according to claim 1, wherein, upon lifting of the femoral head from the acetabular liner, the capsule applies pressure on the femoral head to return it to the acetabular liner.

16. A hip prosthesis system comprising a capsule according to claim 1 and an acetabular liner.

17. The hip prosthesis system according to claim 16 wherein the capsule and acetabular liner are joined at mating region.

18. The hip prosthesis system according to claim 17 wherein the capsule and acetabular liner are joined using an adhesive.

19. The hip prosthesis system according to claim 18 wherein the adhesive is a biodegradable adhesive.

20. The hip prosthesis system according to claim 18 wherein the adhesive is selected from the group consisting of: a polyurethane adhesive, cyanoacrylate adhesive, an ultraviolet-activated adhesive, an acrylic adhesive, a silicone adhesive and an epoxy adhesive.
